(19) United States
(12) Patent Application Publication
(10) Pub. No.: US 2025/0290032 A1
(43) Pub. Date: Sep. 18, 2025

(54) DEVICE AND METHODS FOR ENGINEERING AND MEASURING FLATTENED 3D CELL CULTURES

(71) Applicant: The Trustees of Indiana University, Bloomington, IN (US)

(72) Inventors: Feng Guo, Bloomington, IN (US); Hongwei Cai, Bloomington, IN (US); Zheng Ao, Bloomington, IN (US)

(73) Assignee: The Trustees of Indiana University, Bloomington, IN (US)

(21) Appl. No.: 18/861,349

(22) PCT Filed: Apr. 28, 2023
(86) PCT No.: PCT/US2023/066364

§ 371 Date: Oct. 29, 2024

Related U.S. Application Data
(60) Provisional application No. 63/336,453, filed on Apr. 29, 2022.

(51) Int. Cl. C12N 5/00; C12M 1/00; C12M 1/04; C12M 1/32; C12M 1/42; C12M 3/06; C12N 5/071; C12N 5/079; C12N 5/09; G01N 33/483; G01N 33/50; G01N 33/53; G01N 33/58

(52) U.S. Cl. CPC C12N 5/0062; C12M 23/12; C12M 23/16; C12M 23/24; C12M 29/04; C12M 35/02; C12N 5/0618; C12N 5/0693; C12N 5/0697; G01N 33/4833; G01N 33/5005; G01N 33/53; G01N 33/582; C12N 2513/00

(57) ABSTRACT

The invention relates to improved methods for growing and measuring flattened 3D cell cultures, to the 3D printed scaffolds involved in said methods, and to uses of said 3D cell cultures.

### FIELD

This disclosure relates generally to engineering and measuring flattened 3D cell cultures, and more particularly to developing devices and methods for constructing said 3D cell cultures and using them to model pathological and physiological conditions.

### BACKGROUND

Organoids are tiny, self-organized three-dimensional tissue cultures that are derived from stem cells. They may be fashioned to replicate much of the complexity of an organ, or to express selected aspects of it. The ability of stem cells to self-renew and give rise to specialized progeny allows them to develop into to complex structures. The self-organizing potential of stem cells is showcased by these organoids which capture multiple histological and functional aspects of real organs with fidelity unmatched by previous in vitro models.

Organoids can be formed in vitro by the proliferation and differentiation of tissue-specific cells within a 3D matrix scaffold. Differentiation of cells within these types of scaffolds has been met with varied success.

The ability to generate organoids from adult or induced pluripotent human stem cells has afforded previously unimaginable possibilities for modeling human development, disease, and drug development. Moreover, organoids promise to significantly advance the fields of tissue engineering and cell-based therapies, by serving as sources of highly organized and functional tissue for the repair of damaged or diseased organs.

### SUMMARY OF THE DISCLOSURE

In a first example of the present disclosure, a method for growing a flattened organoid is disclosed. The method can include 3D printing a microfluidic device with a gas permeable membrane attached, wherein the 3D printed microfluidic device is capable of restricting organoid growth to be less than 1000 micrometres in thickness, seeding one or more self-renewing cells capable of differentiating to form an organoid into the microfluidic device, and culturing the colony under differentiation conditions such that the colony undergoes morphogenesis to form an organoid.

In a second example of the present disclosure, a 3D printed microfluidic device is provided. The device includes a gas permeable membrane for fabricating flattened spinal cord organoids that are less than 1000 micrometres in thickness.

In a third example of the present disclosure, a method for modelling opioid induced hyperalgesia is disclosed. The method involves growing a flattened organoid, confirming the presence of neuron populations in said organoid, administering capsaicin to the flattened organoid, measuring the mean firing rate of the organoid, administering the opioid DAMGO, and then finally measuring the mean firing rate of the organoid.

### BRIEF DESCRIPTION OF THE DRAWINGS

FIG. 1 illustrates (a) an image of the 24 well plate with flattened organoid devices inserted, (b) a schematic of the microfluidic device for generating the organoids, (c) schematics of the flattened spinal organoids growing, receiving pain stimulator, and opioids, and (d) an illustration of electrical signals from flattened spinal cord organoids after receiving different treatments.

FIG. 2 illustrates (a) schematics and images of flattened spinal cord organoids fabrication in the microfluidic devices and (b) Visualization of spinal cord organoids' hypoxia at 60 days in culture by conventional and flattened culture. Scale bar=500 μm.

FIG. 3 illustrates (a) Brightfield images of traditional spherical spinal cord organoids (Ctrl) and flattened spinal cord organoids (Flattened) on the MEA plates, (b) representative raster plots of the MEA signals from traditional organoids and flattened organoids, (c) quantification of active electrodes in the electrical recording of traditional organoids and flattened organoids. Scale bar=500 μm.

FIG. 4 illustrates (a) representative raster plots of the MEA signals from flattened organoids at different developing stages and (b-e) quantification (burst frequency, firing rate, synchrony index and network burst frequency) of electrical activities from flattened spinal cord organoids at different stages. Scale bar=500 μm.

FIG. 5 illustrates (a) immunofluorescence showing the expression of u-opioid receptors on sensory neurons (CGRP), GABAergic interneurons and glutamate interneurons, (b) representative raster plots showing the MEA signals of flattened spinal cord organoids treated with capsaicin only and capsaicin and DAMGO together, (c) mean firing rates change from baseline (%) of flattened spinal cord organoids treated with pain stimulator only and pain stimulator together with different doses of DAMGO. Scale bar=50 μm.

FIG. 6 illustrates (a) mean firing rates change from baseline (%) of flattened spinal cord organoids treated with pain modulator under administration of vehicle (Ctrl), 500 nM DAMGO (DAMGO) and 500 nM DAMGO together with 1 μM Naloxone at different time stages, (b) after 8-day administration, mean firing rates change from baseline (%) of the three groups of flattened spinal cord organoids treated with pain stimulator only and pain stimulator together with different doses of DAMGO, (c) immunofluorescence showing the expression of u-opioid receptors after 8-day administration vehicle (Ctrl), 500 nM DAMGO (DAMGO) and 500 nM DAMGO together with 1 μM naloxone, (d) quantification of μ-opioid receptors mean fluorescent intensity for the three groups.

### DETAILED DESCRIPTION

More than 20% of the general population suffers from chronic pain. Due to the increasing onset of chronic pain and its heavy burden on patients, there has been increasing interest in studying pain treatment and management. Among the various pain relief treatments, opioids are among the largest and fastest growing class of medications prescribed by physicians in the United States. However, intended administration of opioids for chronic pain induces undesirable side effects such as opioid-induced hyperalgesia (OIH) and opioid tolerance. These drawbacks represent a major concern when using opioids and may reduce the pain relief efficacy of opioids over time.

Recent developments of human stem cell culture and differentiation technology shows a promising potential for human based in vitro pain models. Protocols have been established to study stem cell derived sensory neurons as well as sensory ganglion organoids. However, these sensory neuron cultures lack the critical spinal cord dorsal horn neurons that are key to understand pain circuitry. Protocols to generate spinal cord organoids with dorsal interneurons and sensory neurons were developed. However, the electrical activity of dorsal spinal cord organoids has not yet been explored, nor have they been adopted as models for pain etiology and opioid responses. These deficiencies are likely due to the lack of integration of electrical measuring methods with good throughput and standardization. This disclosure addresses this and other needs

To overcome these barriers, this present disclosure provides methods for fabricating a human spinal cord organoid model with abundant neural activity and functionalities to study pain and opioid interactions. The device comprises a 3D printed organoid holder with a gas permeable membrane to allow for organoid growth. In some embodiments, the 3D printed holder restricts organoid growth to be less than 500 micrometers in thickness which prevents necrosis and hypoxia. This restriction also helps limit the organoid morphology to further decrease organoid heterogeneity. Additionally, organoid growth on the gas permeable membrane allows “plug and play” features for electrophysiology measurements, which allows for repeatable sampling with longitudinal monitoring of organoid electrical activity, as well as easy access to study their responses to various modulators.

As used in the present disclosure, the term “3D cell culture” refers to an artificially grown mass of cells or tissue. The term “organoid” as used herein denotes a miniaturized version of an organ produced in vitro in 3 dimensions that shows realistic micro-anatomy.

According to one embodiment of the present disclosure, a method for growing a flattened 3D cell culture is provided, comprising:
(i) 3D printing a microfluidic device with a gas permeable membrane attached, wherein the 3D printed microfluidic device is capable of restricting 3D cell culture growth to be less than 1000 micrometres in thickness;
(ii) seeding one or more self-renewing cells capable of differentiating to form a 3D cell culture into the microfluidic device; and
(iii) culturing the colony under differentiation conditions such that the colony undergoes morphogenesis to form a 3D cell culture.

In one embodiment, the 3D printed microfluidic device may be of any desired shape or size, preferably wherein the structure is capable of restricting 3D cell culture growth to a thin sheet. Preferably, the structure may comprise cavities arranged in an array. The array may be an ordered arrangement of similar or identical wells which is typically divided into rows and columns. Preferably, the cavity has a 3D structure comprising a cylinder, preferably wherein the cylinder has an outer diameter of 15 mm, an inner diameter of 11 mm and a height of 5 mm.

In one embodiment, the 3D printed microfluidic device is used in integration with a multiwell microelectrode assay system or a multiwell flat bottom cell culture plate. For example, a 24 or 96 well flat bottom cell culture plate or 24 well MEA system which are commercially available from Corning.

In one embodiment, the 3D structure is capable of restricting 3D cell culture growth in a well to be less than 1000 micrometers in thickness, less than 900 micrometers in thickness, less than 800 micrometers in thickness, less than 700 micrometers in thickness, less than 600 micrometers in thickness, less than 500 micrometers in thickness, less than 400 micrometers in thickness, less than 300 micrometers in thickness, less than 200 micrometers in thickness, less than 100 micrometers in thickness, or less than 50 micrometers in thickness.

The method of producing the 3D printed microfluidic device may involve replica molding, soft embossing, injection molding, 3D printing, bioprinting, laser machining, micromachining, surface etching, optical lithography, additive manufacturing, electrochemical directed crosslinking soft-lithography, and/or polydimethyl siloxane (PDMS) replica molding.

In one embodiment, the self-renewing cells are stem cells or tumor cells, preferably embryonic, induced pluripotent, small intestinal, stomach, colon, pancreatic, liver, lunch, prostate, mammary, corneal, hair follicle, epidermal, or kidney cells, or progenitors of such cells.

The 3D printed microfluidic device may comprise a polycarbonate membrane. The polycarbonate membrane may contact the surface of the 3D cell culture as it grows, allowing it to expand and differentiate while maintaining a uniform thickness.

The substrate within the wells may be a hydrogel. The hydrogel of the invention may be formed of macromolecules of natural origin and selected from the group comprising polysaccharides, gelatinous proteins, agarose, alginate, chitosan, dextran, laminins, collagens, hyaluronan, fibrin or mixtures thereof, or are selected from the group of complex tissue-derived matrices consisting of Matrigel, Myogel and Cartigel.

In one embodiment, the self-renewing cells are cultured in the microfluidic device to form a 3D cell culture. Culturing the cells in an expansion medium allows the cells to multiply whilst retaining their stem or progenitor cell phenotype. The components to promote differentiation conditions may comprise factors previously described to be necessary for culturing stem cell colonies of different origins in contact with an extracellular matrix, such as a BMP inhibitor, a Wnt agonist and Epidermal Growth Factor, added to a basal medium for animal or human cells culture. Differentiation conditions according to the invention may comprise factors previously described to be necessary for culturing and obtaining 3D stem cell cultures including embryonic body medium, spinal cord medium II, spinal cord medium III, and spinal cord medium IV.

In on embodiment, human spinal cord organoids may be fabricated by aggregation of approximately 9000 WA09 cells per EB using a 96-well spheroid microplate (Corning). Embryonic bodies (EBs) may be formed in 100 μL Aggrewell EB formation medium (Stemcell Technologies) supplemented with 10 UM Y-27632 (SelleckChem). After the EB formation (Day 1), the medium may be switched to the spinal cord medium I (ScM I) containing 3 UM CHIR-99021 (Stemcell Technologies) and 10 nM retinoic acid (RA) (Sigma-Aldrich). After 4 days of culturing in in ScM I, the medium may then be switched to ScM II containing 5 ng/ml recombinant human BMP4 (Peprotech) and 10 nM retinoic acid to continue culturing for 6 days. On day 10, spinal cord organoids may be embedded in Matrigel (Corning). The medium may be switched to ScM III containing 10 μM N-[N-(3,5-difluorophenacetyl)-Lalanyl]-S-phenyl glycine t-butyl ester (DAPT) and kept for 8 days. During this period, spinal cord organoids may be transferred to six-well ultralow attachment plates (Corning) held on an orbital shaker (Benchmark) set at 60 rpm. On Day 18, the medium may belly switched to ScM IV, containing 20 μg/mL ascorbic acid (Sigma-Aldrich) and 1 μM cyclic adenosine monophosphate (cAMP) (Sigma-Aldrich) for continuous culture on the orbital shaker. During this process, the medium may be refreshed every other day.

Another aspect of the invention relates to a method for modelling opioid induced hyperalgesia comprising:
(i) growing a flattened 3D cell culture;
(ii) confirming the presence of neuron populations in the flattened 3D cell culture by immunofluorescence staining;
(iii) administering capsaicin to the flattened 3D cell culture;
(iv) measuring the mean firing rate of the 3D cell culture;
(v) administering DAMGO; and
(vi) measuring the mean firing rate of the 3D cell culture.

Growth of the 3D cell culture may be done using the methods described herein using the 3D printed microfluidic device. Preferably, the 3D cell culture is flattened with a thickness of 1000 micrometers or less.

Immunofluorescence staining may be used to characterize the cultured 3D cell cultures. The staining may be carried out according to any suitable protocol such as first washing with PBS, HCl, followed by antibody incubation and mounting using anti-fade mounting media such as those containing DAPI (Invitrogen).

To evaluate the response of the flattened spinal cord 3D cell cultures to pain modulators and opioid treatments, a capsaicin and DAMGO may be used to model pain responses. Capsaicin, a transient receptor potential cation channel subfamily V member 1 (TRPV1) agonist may be applied to the 3D cell cultures to model pain responses. In some embodiments, the stimulation by capsaicin may be followed by different concentrations of an opioid to test pain relief effects. A suitable opioid is DAMGO which may be administered in different concentrations such as 10 μM or less, 1 μM or less, 500 nM or less, or 100 nM or less.

To measure the mean firing rate of the 3D cell cultures, electrical activity may be measured according to any suitable method. For example, the recording of electrical activity may be performed by using the Axion Mestro Edge (Axion inc). To minimize statistical variation, a final mean value may be calculated based on the mean firing rate of several recordings.

To measure hypoxic core formation and evaluate necrosis, the 3D cell cultures as described herein may be stained with a fluorescent hypoxia indicator. To visualize hypoxic core formation within the engineered 3D cell cultures, samples may be subjected to staining by the Image-iT red hypoxia kit (Invitrogen). Samples maybe incubated with the Image-iT red hypoxia dye for 4 hours before imaging on an inverted fluorescence microscope (Olympus IX-83). Compared to other methods, the hypoxic core formation of the 3D cell cultures produced in accordance with the present disclosure may be drastically reduced

Another aspect of the invention relates to a method for producing an in vivo assay of a 3D cell culture comprising:
(i) growing a flattened 3D cell culture;
(ii) attaching the flattened 3D cell culture to a microelectrode array system; and
(iii) recording the electrical activity of the 3D cell cultures.

Growth of the 3D cell culture may be done using the methods described herein using the 3D printed microfluidic device. Preferably, the 3D cell culture is flattened with a thickness of 1000 micrometers or less.

The electrical activity of the 3D cell cultures may be measured using the microelectrode array system such as the Axion Maestro Edge (Axion inc.). In one embodiment, the mean firing rate of the 3D cell cultures is measured.

Another aspect of the invention relates to a method for optically imaging a 3D cell culture comprising:
(i) growing a flattened 3D cell culture according to the method of claim 1;
(ii) imaging the 3D cell culture using an imaging technique selected from the group consisting of brightfield microscopy, phase-contrast microscopy, fluorescent imaging, and confocal microscopy.

The imaging of the 3D cell cultures may be conducted on a microelectrode array plate. The constrained thickness of the 3D cell culture may promote clearer imaging and better visibility of internal cellular processes.

As such, whereas particular embodiments of this invention have been described above for purposes of illustration, it will be evident to those skilled in the art that numerous variations of the details of the present invention may be made without departing from the invention as defined in the appended claims.

The following examples serve to further illustrate the disclosure as described herein, and are not intended to limit the scope of the claims.

### EXAMPLES

### Example 1: 3D Printed 3D Cell Culture Holder

To minimize necrosis and maintain neuron functions to measure the change in pain and substance related activity, a microfluidic device for flattened 3D cell culture fabrication was developed. The device consists of a 3D printed hollow ring with an outer diameter of 15 mm, an inner diameter of 11 mm, and a height of 5 mm. The 3D cell culture holder device was designed in AutoCAD software with the desired dimensions. The device was then printed using a stereolithography 3D printer (Form 3B, Formlabs) using the FormLabs Clear Resin V4 (FormLabs) printing material. The device was then attached to a gas permeable polycarbonate membrane.

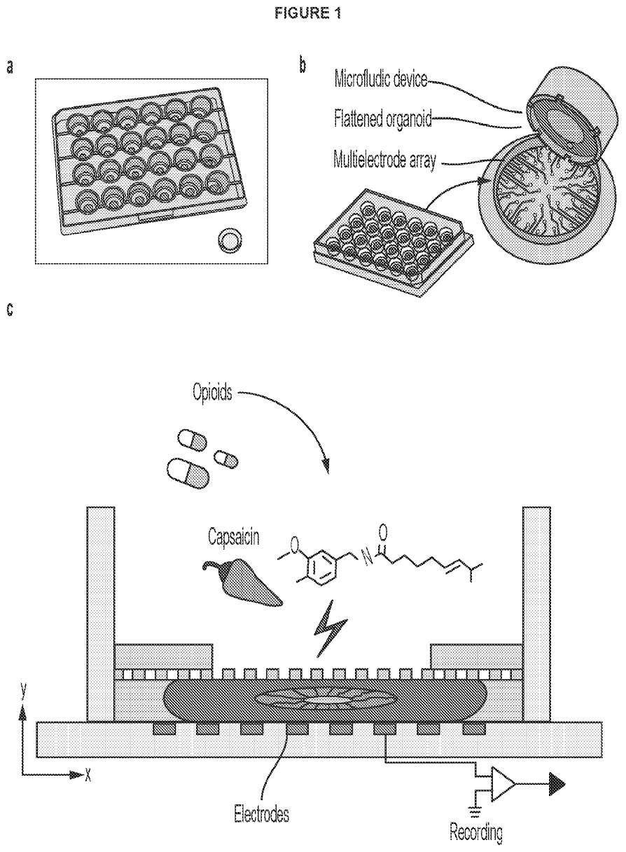

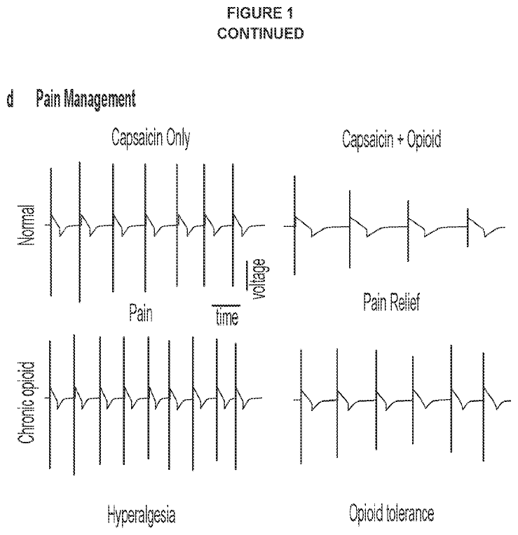

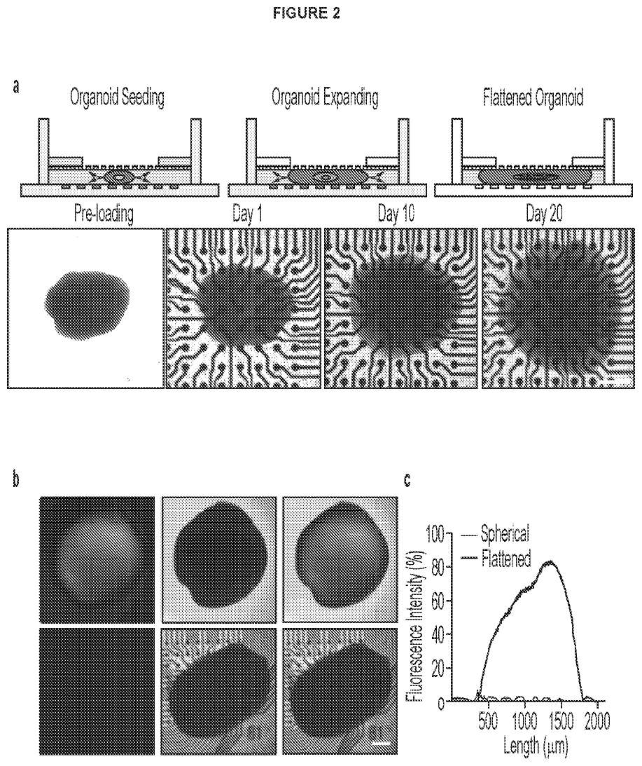

The 3D printed device is inserted into 24 well plates or 24 well MEA systems which are illustrated in FIG. 1a.

### Example 2: Fabrication of Flattened Spinal Cord 3D Organoids and Hypoxic Characterization

Human embryonic stem cell WA09 was obtained from WiCell institute and guidelines of both WiCell institute and Indiana University were observed closely when handling these cells. Matrigel (Corning) coated 6 well plates were used to culture WA09 cells with mTESR plus medium (Stemcell Technologies) in an incubator at 37 degree celsius and 5% CO2. Medium was changed every other day. ReLeSR (Stemcell Technologies) was used to passage WA09 cells every week.

Embryonic bodies (EBs) were fabricated using a 96-well U bottom microplate (Corning) by aggregation of ˜9,000 WA09 cells in each well. EBs were cultured in 100 μL EB formation medium (Stemcell Technologies) supplemented with 10 μM Y-27632 (SelleckChem). After the EB formation (Day 1), the EBs were switched to the spinal cord medium I (ScM I) containing 10 nM retinoic acid (Sigma Aldrich) and 3 μM CHIR-99021 (Stemcell Technologies). After 4-days culture in ScM I, the spheroids were then transferred to spinal cord medium II (ScM II) with 10 nM retinoic acid and 5 ng/ml recombinant human BMP4 (Peprotech) for the next 6 days. On day 10, the spinal cord organoids were embedded into Matrigel (corning). The organoids were switched to spinal cord medium III (ScM III) and supplemented with 10 μM DAPT and cultured for the next 8 days. During this period, spinal cord organoids were transferred to the above-described microfluidic devices integrated with 24 well ultra-low attachment plates (Corning) or 24 well MEA systems for flattened organoids generation. For traditional spherical organoids, the organoids were transferred to a 6 well ultra-low attachment plates (Corning) shaking at 60 RPM with an orbital shaker. On Day 18, the organoids were finally transferred to spinal cord medium IV (ScM IV), with 20 μg/mL asorbic acid (Sigma Aldrich) and 1 μM cAMP (Sigma Aldrich) for subsequent continuous culture. Medium change was performed every other day during this process except when specified.

### Example 3: Neuron Activity of Flattened Organoids

After 10 days of growing, the flattened organoids as developed in accordance with the present disclosure were put into contact with bottom electrodes for measuring electrical and neuron activity. Recording of the spinal cord organoids' electrical activity was performed by using the Axion Mestro Edge (Axion inc). The spinal cord organoids were kept at 37° C. maintained by an internal heater and infused with 5% CO2. To determine the mean firing rate of a spinal cord organoid, the 3-minute MEA recording was repeated 5 times. The final mean firing rate of the organoid was calculated as the average mean firing rate of the 3 most consistent recordings among the 5 recordings. To reduce the variation brought by adding substance, the medium was also refreshed before measuring mean firing rate baseline. The substance was first diluted into ScM IV at 2× of the desired concentration, then half of the medium was replaced with the 2× concentrated solution to reach the desired concentration.

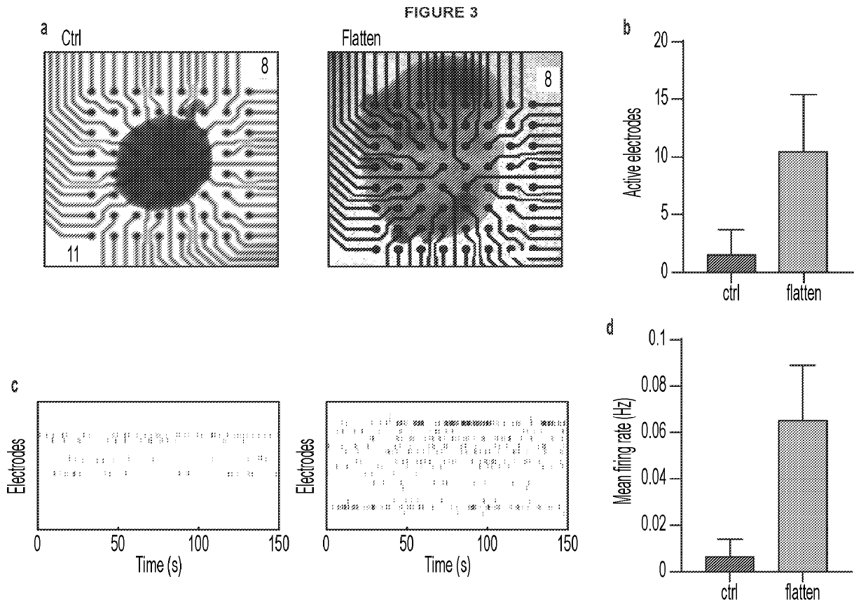

FIG. 3b illustrates how the active electrodes of the flattened organoids were significantly more abundant than spherical organoids. This abundance promotes better MEA measurement conditions and results. The electrical activity of the flattened organoids was measured and is shown in Table 1 below.

TABLE 1
Mean firing rate of flattened organoids versus control.

| | Control | Flattened Organoids |
|---|---|---|
| Mean Firing Rate (Hz) | 0.01 | 0.07 |

### Example 4: Pain Relief by Short-Term Opioid Administration

To first validate that the flattened organoids could be utilized to model pain relief, first immunofluorescence staining was used to confirm that the presence of neuron populations that express μ-opioid receptors. Sectioned samples were placed onto charged glass slides and washed three times with 1×PBS. The samples were then treated with 3N hydrochloric acid (HCl) for 15 minutes for antigen retrieval. Following HCl treatment, the samples were washed again twice with 1×PBS and subjected to blocking (0.3% Triton-X100, 5% normal goat serum in 1×PBS) for 1 hour, followed by primary antibody incubation in a humidified chamber at 4° C. overnight. The samples were then washed 3 times with 1×PBS followed by secondary antibody incubation at room temperature for 1 hour before being washed and coverslipped with gold anti-fade mounting medium containing DAPI (Invitrogen).

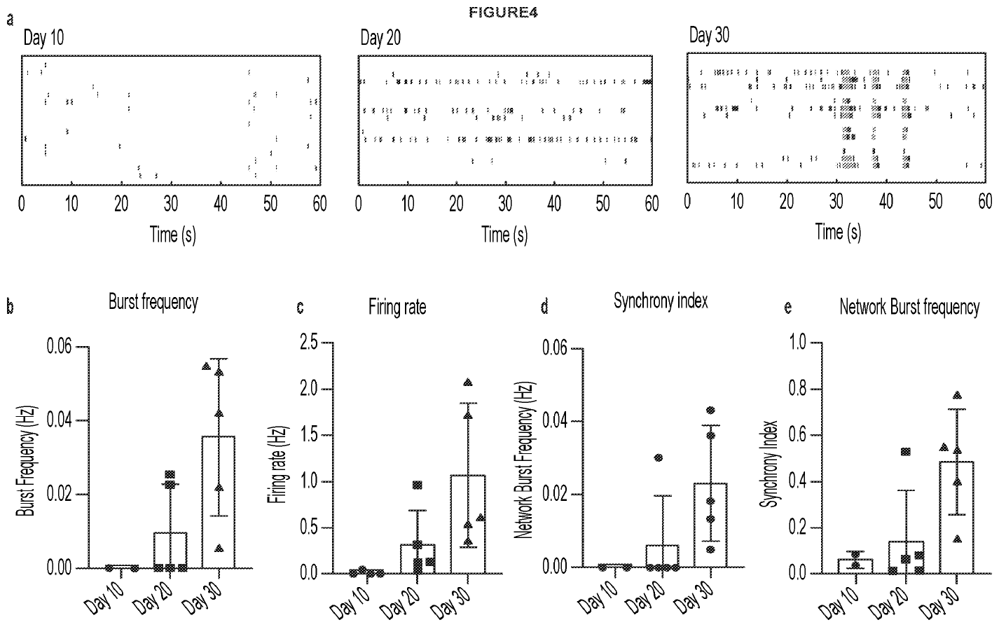

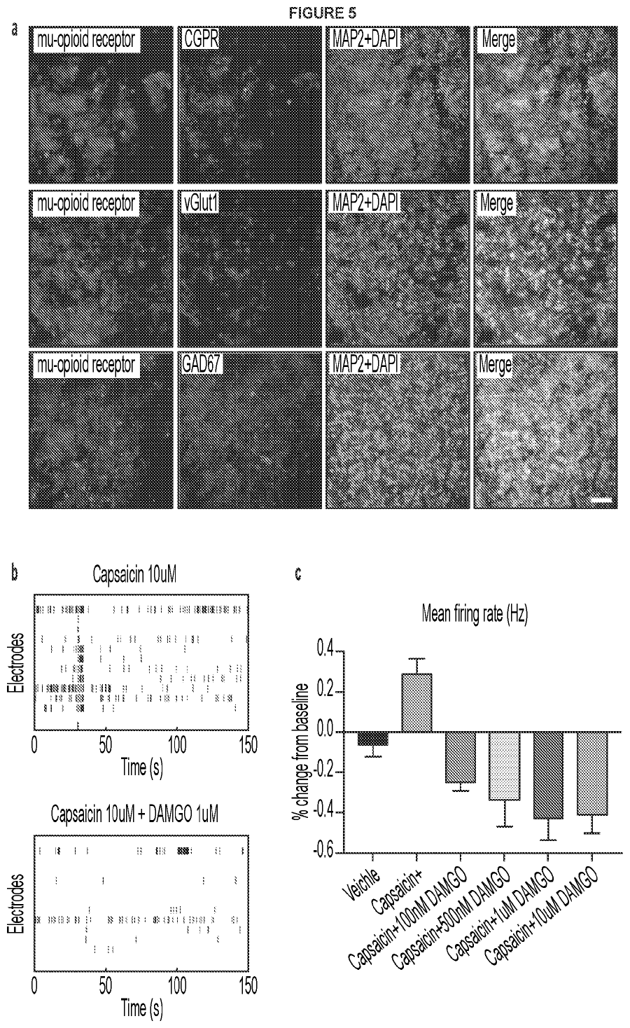

Capsaicin, a transient receptor potential cation channel subfamily V member 1 (TRPV1) agonist, was applied to fSCOs to model pain responses. After stimulation by capsaicin, the mean firing rate of organoids increased 35.4%. Next, capsaicin and different concentrations of DAMGO together to test the pain relief effects of different opioids doses. As shown in FIG. 4c and Table 2 below, the opioids treatment reduced the pain effectively and the firing rate of spinal cord organoids dropped below baseline even under minimal dose of DAMGO (100 nM). As the opioids doses increase, the fSCOs' firing rates decreased, and the organoids reached minimal firing rates when the concentration was higher than 1 μM.

TABLE 2
Quantification of electrical activities from flattened spinal
cord organoids at different stages of development.

| | Burst | Firing | Network Burst | |
|---|---|---|---|---|
| | Frequency | Rate | Frequency | Synchrony |
| | (Hz) | (Hz) | (Hz) | Index |
| Day 10 | 0 | 0 | 0 | 0.05 |
| Day 20 | 0.01 | 0.25 | 0.01 | 0.1 |
| Day 30 | 0.035 | 1.0 | 0.023 | 0.5 |

### Example 5: Modeling Chronic Opioid Induced Hyperalgesia

The fSCOs of the present disclosure were exposed to prolonged opioid administration to test the tolerance of the organoids to opioid induced hyperalgesia. The fSCOs were treated with DAMGO (500 nM) for 8 days and their responses to pain modulators were tested every other day. In the first 4 days, the DAMGO treated group and the control group showed no significant difference of mean firing rate increase after capsaicin treatment. DAMGO treated organoids showed significantly heightened firing rate increase on day 6 (73% versus 48%) and day 8 (93% versus 39%), indicating a heightened pain sensitivity to capsaicin. Organoids treated together with DAMGO and Naloxone, a μ-opioid receptor antagonist, were also tested to determine whether the Naloxone could reduce the opioid induced hyperalgesia. The DAMGO+Naloxone group showed no significant difference on capsaicin induced mean firing change, indicating reduced opioid induced hyperalgesia.

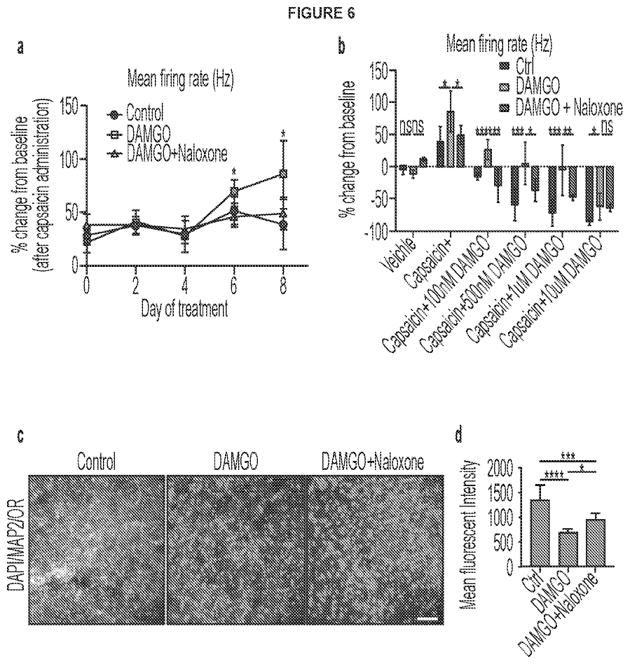

Table 3 provides the fSCOs' responses to capsaicin and different doses of DAMGO. A low dose of DAMGO (100 nM) was able to reduce the firing rate below baseline and relieve the pain activity induced by capsaicin in Ctrl and DAMGO+Naloxone group, while organoids in the DAMGO group needed more than 1 μM of DAMGO to eliminate pain activity and return to activity baseline. To further explore if the fSCOs can recapitulate the key receptors and pathological changes of in vivo animals models with prolonged opioid administration, immunofluorescence staining was performed on these three groups of organoids (FIG. 6c). μ-opioid receptor expression was downregulated in the prolonged DAMGO treatment group.

TABLE 3
Mean firing rates for fSCOs with different treatments.

| | | Capsa- | Capsa- | Capsa- | Capsa- |
|---|---|---|---|---|---|
| | | icin + | icin + | icin + | icin + |
| | Capsa- | 100 nM | 500 nM | 1 uM | 10 uM |
| | icin | DAMGO | DAMGO | DAMGO | DAMGO |
| % change From | −0.1 | +0.3 | −0.2 | −0.42 | −0.4 |
| Baseline in | | | | | |
| Mean Firing | | | | | |
| Rate (Hz) | | | | | |

1. A method for growing a flattened 3D cell culture comprising:
(i) 3D printing a microfluidic device with a gas permeable membrane attached, wherein the 3D printed microfluidic device is capable of restricting the 3D cell culture's growth to be less than approximately 1000 micrometres in thickness;
(ii) seeding one or more self-renewing cells capable of differentiating to form a 3D cell culture into the microfluidic device; and
(iii) culturing the colony under differentiation conditions such that the colony undergoes morphogenesis to form a 3D cell culture.

2. The method of claim 1, wherein the 3D printed microfluidic device comprises a hollow ring with an outer diameter of approximately 15 mm, an inner diameter of approximately 11 mm, and a height of approximately 5 mm.

3. The method of claim 1, wherein the gas permeable membrane is a polycarbonate membrane.

4. The method of claim 1, wherein the 3D printed microfluidic device is used in integration with a multiwell microelectrode array system.

5. The method of claim 1, wherein the self-renewing cells are stem cells or tumour cells and/or the self-renewing cells are embryonic, induced pluripotent, small intestinal, stomach, colon, pancreatic, liver, lung, prostate, mammary, corneal, hair follicle, epidermal or kidney cells or progenitors of such cells.

6. The method of claim 1, wherein the flattened 3D cell culture is a spinal cord organoid.

7. The method of claim 1, wherein the hypoxic core formation of the flattened 3D cell culture is less than the hypoxic core formation of flattened 3D cell cultures produced by traditional non-flattened 3D cell cultures as measured by a fluorescent hypoxia indicator.

8. A 3D printed microfluidic device with a gas permeable membrane for fabricating a flattened 3D cell culture, wherein the 3D printed microfluidic device is capable of restricting the 3D cell culture's growth to be less than approximately 1000 micrometres in thickness.

9. The device of claim 8, wherein the 3D printed microfluidic device comprises a hollow ring with an outer diameter of approximately 15 mm, an inner diameter of approximately 11 mm, and a height of approximately 5 mm.

10. The device of claim 8, wherein the gas permeable membrane is a polycarbonate membrane.

11. The device of claim 8, wherein the device further comprises an multiwell microelectrode array system.

12. A method for modelling opioid induced hyperalgesia comprising:
(i) growing a flattened 3D cell culture according to the method of claim 1;
(ii) confirming the presence of neuron populations in the flattened 3D cell culture by immunofluorescence staining;
(iii) administering capsaicin to the flattened 3D cell culture;
(iv) measuring the mean firing rate of the 3D cell culture;
(v) administering DAMGO; and
(vi) measuring the mean firing rate of the 3D cell culture.

13. A method for producing an in vivo assay of a 3D cell culture comprising:
(i) growing a flattened 3D cell culture according to the method of claim 1;
(ii) attaching the flattened 3D cell culture to a microelectrode array system; and
(iii) recording the electrical activity of the 3D cell culture.

14. The method of claim 13, wherein the electrical activity is the mean firing rate.

15. A method for optically imaging a 3D cell culture comprising:
(i) growing a flattened 3D cell culture according to the method of claim 1;
(ii) imaging the 3D cell culture using an imaging technique selected from the group consisting of brightfield microscopy, phase-contrast microscopy, fluorescent imaging, and confocal microscopy.
